(19) United States
(12) Patent Application Publication
(10) Pub. No.: US 2023/0381385 A1
(43) Pub. Date: Nov. 30, 2023

(54) TECHNIQUES FOR DETERMINING ACID-BASE HOMEOSTASIS

(71) Applicants: Fresenius Medical Care Holdings, Inc., Waltham, MA (US); Fresenius Medical Care Deutschland, GMBH, Bad Homburg (DE)

(72) Inventors: Alhaji Cherif, Kew Gardens, NY (US); Paulo Paneque Galuzio, New York, NY (US); Peter Kotanko, New York, NY (US); Juergen Klewinghaus, Bad Homburg (DE); David Thompson, Waltham, MA (US)

(21) Appl. No.: 18/201,396

(22) Filed: May 24, 2023

Related U.S. Application Data
(60) Provisional application No. 63/345,344, filed on May 24, 2022.

(51) Int. Cl. A61M 1/16

(52) U.S. Cl. CPC A61M 1/1613; A61M 2230/202

(57) ABSTRACT

The described technology may include processes to model acid-base homeostasis in normal patients and under acid-base disorder conditions. In one embodiment, a method may include an acid-base homeostasis analysis process. The method may include, via a processor of a computing device, providing an acid-base model configured to model acid-base homeostasis of a patient, the acid-base model comprising a patient model, a dialyzer model, and an extracorporeal CO₂ removal device (ECCO₂RD), and determining predicted patient information using the acid-base model. Other embodiments are described.

### CROSS-REFERENCE TO RELATED APPLICATION

This application claims the benefit to U.S. Provisional Application No. 63/345,344, filed May 24, 2022, the entire contents of which are incorporated herein by reference in their entirety.

### FIELD

The disclosure generally relates to processes for modeling the functionality of portions of the human body to generate healthcare information, treatment recommendations, and/or the like and, more particularly, to techniques for modeling acid-base homeostasis of a patient being treated via certain medical devices, such as a dialyzer and/or an extracorporeal CO2 removal device (ECCO₂RD).

### BACKGROUND

Precise maintenance of pH and acid-base homeostasis is fundamental for the optimal functioning of physiological and cellular functions. The presence of an acid-base disturbance can affect clinical outcomes and may be associated with an underlying disease. Acid-base disturbances are common in patients with renal health complications, such as dialysis patients. Continuous renal replacement therapy (CRRT) is a method of slower, continuous dialysis that facilitates solute and fluid homeostasis. Acid-base complications can be a contributing factor to morbidity of CRRT patients. Accordingly, it is important to assess the acid-base status of patients and the extent to which various therapeutic treatments are effective in controlling these acid-base alterations.

However, conventional methods and technologies are not able to determine patient information sufficient to accurately and effectively provide decision support to guide clinicians in determining treatments nor setting optimal parameters for selected treatment regimens.

It is with respect to these and other considerations that the present improvements may be useful.

### SUMMARY

This Summary is provided to introduce a selection of concepts in a simplified form that are further described below in the Detailed Description. This Summary is not intended to necessarily identify.

In an embodiment, an apparatus may include at least one processor and a memory coupled to the at least one processor, the memory including instructions that, when executed by the at least one processor, cause the at least one processor to access an acid-base model configured to model acid-base homeostasis of a patient, the acid-base model comprising a patient model, a dialyzer model, and an extracorporeal CO₂ removal device (ECCO₂RD) model, and determine predicted patient information using the acid-base model.

In some embodiments of the apparatus, the predicted patient information may include at least one of a blood flow rate (Q), a serum pH level, a pCO₂ level, or a HCO₃ level.

In some embodiments of the apparatus, the predicted patient information may include a treatment recommendation. In various embodiments of the apparatus, the treatment recommendation may include a treatment process for an acid-base disorder.

In some embodiments of the apparatus, the instructions, when executed by the at least one processor, may cause the at least one processor to determine continuous renal replacement therapy (CRRT) parameters to control acid-base status based on the predicted patient information.

In various embodiments of the apparatus, the acid-base model may be configured to model the regulation of H⁺, CO₂, and HCO₃⁻

In some embodiments of the apparatus, the patient model may be configured to model patient physiology having input of blood flow and output of hydrogen ion concentration, carbon dioxide concentration, and bicarbonate concentration.

In various embodiments of the apparatus, the dialyzer model may be configured to model continuous renal replacement therapy (CRRT).

In exemplary embodiments of the apparatus, the ECCO₂RD model may be configured to model a one-dimensional (1D) diffusion device between blood and air.

In some embodiments of the apparatus, the acid-base model may include a blood flow circuit flowing from a patient, modeled by the patient model, to a dialyzer, modeled by the dialyzer mode, to an ECCO2RD, modeled by the ECCO2RD model, and back to the patient.

In various embodiments of the apparatus, the blood circuit may include diffusion at any point in the blood circuit.

In some embodiments of the apparatus, the patient may include a virtual patient.

In an embodiment, a method, such as a computer-implemented method via a processor of a computing device, of acid-base homeostasis analysis may include, providing an acid-base model configured to model acid-base homeostasis of a patient, the acid-base model comprising a patient model, a dialyzer model, and an extracorporeal CO₂ removal device (ECCO₂RD) model, and determining predicted patient information using the acid-base model.

In some embodiments of the method, the predicted patient information may include at least one of a blood flow rate (Q), a serum pH level, a pCO₂ level, or a HCO₃ level.

In some embodiments of the method, the predicted patient information may include a treatment recommendation. In various embodiments of the of the computer-implemented method, the treatment recommendation may include a treatment process for an acid-base disorder.

In some embodiments of the method, the method may include prescribing continuous renal replacement therapy (CRRT) parameters to control acid-base status based on the predicted patient information.

In some embodiments of the method, the acid-base model configured to model the regulation of H⁺, CO₂ and HCO₃⁻.

In some embodiments of the method, the patient model may be configured to model patient physiology having input of blood flow and output of hydrogen ion concentration, carbon dioxide concentration and bicarbonate concentration.

In some embodiments of the method, the dialyzer model may be configured to model continuous renal replacement therapy (CRRT).

In some embodiments of the method, the ECCO2RD model may be configured to model a one-dimensional (1D) diffusion device between blood and air.

In some embodiments of the method, the acid-base model may include a blood flow circuit flowing from a patient, modeled by the patient model, to a dialyzer, modeled by the dialyzer mode, to an ECCO2RD, modeled by the ECCO2RD model, and back to the patient.

In some embodiments of the method, the blood circuit may include diffusion at any point in the blood circuit.

In some embodiments of the method, the patient may include a virtual patient.

In various embodiments of the method, the predicted patient information may include a treatment recommendation. In some embodiments of the method, the treatment recommendation may include a treatment process for an acid-base disorder.

### BRIEF DESCRIPTION OF THE DRAWINGS

By way of example, specific embodiments of the disclosed machine will now be described, with reference to the accompanying drawings, in which:

FIG. 1 illustrates an example of an operating environment 100 that may be representative of some embodiments in accordance with the present disclosure.

FIG. 2 illustrates an example of an operating environment that may be representative of some embodiments in accordance with the present disclosure.

FIG. 3 illustrates an example of a patient model that may be representative of some embodiments in accordance with the present disclosure.

FIG. 4 illustrates an example table of constant data for acid-base models that may be representative of some embodiments in accordance with the present disclosure.

FIG. 5 illustrates a block diagram of an intradialytic acid-base model that may be representative of some embodiments in accordance with the present disclosure.

FIG. 6 illustrates a table of a first illustrative set of acid-base model parameters that may be representative of some embodiments in accordance with the present disclosure.

FIG. 7 illustrates a table of a second illustrative set of acid-base model parameters that may be representative of some embodiments in accordance with the present disclosure.

FIGS. 8A and 8B illustrate results of a simulation of an acid-base model that may be representative of some embodiments in accordance with the present disclosure.

FIG. 9 depicts an illustrative ECCO₂RD model that may be representative of some embodiments in accordance with the present disclosure.

FIG. 10 illustrates an example table of parameter data for acid-base models that may be representative of some embodiments in accordance with the present disclosure.

FIGS. 11A-11D illustrate results of a simulation of an acid-base model that may be representative of some embodiments in accordance with the present disclosure

FIG. 12 illustrates results of a simulation of an acid-base model that may be representative of some embodiments in accordance with the present disclosure

FIG. 13 illustrates an embodiment of an exemplary computing architecture that may be representative of some embodiments in accordance with the present disclosure.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete and convey the scope of the subject matter to those skilled in the art. In the drawings, like numbers refer to like elements throughout.

Acid-base imbalance is a common complication for patients with chronic kidney disease (CKD), which is typically treated through a dialysis treatment protocol. One type of dialysis treatment protocol is continuous renal replacement therapy (CRRT) which, in general, is a method of slower, continuous dialysis to facilitate solute and fluid homeostasis. Acid-base imbalance, such as acidemia, is a contributing factor to the morbidity of CKD patients, including CRRT patients. The correction, particularly the rapid correction, of acidemia can significantly improve patient outcomes. Therefore, detecting, predicting, and/or correcting acidemia appropriately is of utmost importance.

Accordingly, some embodiments may operate to enable the personalization of treatment for CKD patients, including CRRT patients and/or populations of CRRT patients (for instance, populations with the same or similar characteristics, disease state, etc.). Although CRRT patients are used in some examples in the present disclosure, embodiments are not so limited, as the described processes, techniques, methods, systems, and/or the like may be used for the treatment of other types of CKD patients and/or treatment protocols).

Embodiments may include acid-base homeostasis analysis processes operative to serve as decision support to help clinicians in determining treatments and/or setting optimal treatment parameters for dialysis patients, such as CKD patients, including CRRT patients (with or without mechanical ventilation). The acid-base homeostasis analysis processes may use computational models (“acid-base models” or “models”). The output of the acid-base models may provide guidance in determining parameters, predictions (e.g., patient acid-base characteristics) treatments, treatment recommendations, treatment adjustments, and/or the like. The acid-base homeostasis analysis processes may include the use/analysis of citrate and lactate metabolism, ventilation, and/or other parameters. The acid-base model output may provide prediction of acid-base status in patients, for instance, CKD treatments, including individuals being treated by CRRT (for example, either renal or respiratory replacement therapy).

The use of extracorporeal CO₂ removal devices (ECCO₂RD) in the ICU has been introduced to provide protective ventilation strategies for patients with acute respiratory distress syndrome (ARDS). In general, extracorporeal carbon dioxide removal (ECCO₂R) treatment aims to reduce or even eliminate blood CO₂ to fight against the adverse effects of certain acid-base disorders, such as acidemia. Acute kidney injury (AKI) may develop in patients with ARDS, which may require dialysis treatment. These patients typically undergo CRRT (or RRT), which may include attaching ECCO₂RD to the hemodialysis (HD) circuit as a combined protective ventilation strategy.

Accordingly, some embodiments may incorporate models of a dialyzer and/or of ECCO₂RD into acid-base models. Non-limiting examples of acid-base models may include models the same, similar, and/or adapted from models described in Cherif et al., “A mathematical model of the four cardinal acid-base disorders,” Mathematical Biosciences and Engineering, 17(5):4457-4476 (2020) (“Cherif et al.”) and/or U.S. Patent Application Publication No. 2020/0294676, titled “Techniques for Determining Acid-Base Homeostasis, filed Mar. 11, 2020 (the '676 Publication) (both Cherif et al. and the '676 Publication referred to as the “Acid-Base Dynamics Disclosure”), the contents of both of which are incorporated by reference as if fully set forth in the present disclosure.

Models according to some embodiments may describe the regulation of H+, CO₂ and HCO₃⁻. In various embodiments, models may be configured to capture continuous veno-venous hemodialysis (CVVHD) with a gas exchanger integrated into an HD system, such as a continuous HD system.

In some embodiments, models may be configured to capture different CRRT modalities (e.g., continuous veno-venous hemofiltration (CVVH), CVVHD, continuous veno-venous hemodiafiltration (CVVHDF), and/or the like), for example, with pre- or post-substitution/dilution fluid, with gas exchanger attached pre- or post-filter, and/or the like.

In some embodiments, models may be or may include mathematical models of intradialytic acid-base dynamics in patients subjected to extracorporeal CO₂ removal.

The described technology may generally include an acid-base homeostasis analysis process operative to simulate pH and acid-base homeostasis for one or more patients, one or more patient populations, one or more virtual patients, and/or one or more virtual patient populations. In some embodiments, patients and/or virtual patients may be or may include one or more patients, one or more physiological systems (for instance, renal system, pulmonary system, respiratory system, organs thereof, functions thereof, and/or the like), one or more patient populations, portions thereof, virtual models thereof, and/or the like. In some embodiments, the acid-base homeostasis analysis process may use one or more acid-base models that may include the major physiological components and mechanisms governing pH and acid-base homeostasis in patients, including healthy (or normal) patients, patients with abnormalities or disorders, and/or patients undergoing treatment regimens (for instance, dialysis, such as HD).

In various embodiments, the acid-base models may include one or more physiological acid-base models (for instance, a general model of a patient) and one or more intradialytic acid-base models to simulate dialysis patients and dialyzer operation. In various embodiments, intradialytic acid-base models may include one or more of a dialysis patient model (for instance, a model with impaired functionality corresponding to dialysis patients, such as impaired renal regulation), and a dialyzer model to simulate intradialytic dynamics associated with pH and acid-base homeostasis.

In some embodiments, the acid-base models may operate as a dynamic model of the physiological regulation of a HCO₃/CO₂ buffering system. In various embodiments, the acid-base models may be configured using or based on, at least in part, Henderson-Hasselbalch kinetics. For example, some embodiments may include acid-base models of the HCO₃/CO2 buffering system with Henderson-Hasselbalch mass-action kinetics.

In exemplary embodiments, the acid-base models may simulate a normal physiological state and several acid-base disorders, including, without limitation, metabolic acidosis, metabolic alkalosis, respiratory acidosis, and/or respiratory alkalosis.

Regulation of pH and acid-base homeostasis in the blood and in the extracellular fluid plays a pivotal role in many aspects of cellular metabolism and other physiological functions. The impact of acid-base alterations has far-reaching implications. In addition to physiochemical buffering, acid-base homeostasis is regulated by respiratory and renal systems. Changes in pH affect numerous physiochemical reactions and buffering systems, transport/channel kinetics, muscle contraction, metabolic enzymatic reactivities, and protein/membrane structures and functions. Alterations in pH also impact other functions, including cardiovascular, central nervous, renal and pulmonary systems, tissue metabolism and oxygenation, and bone remodeling. For instance, chronic H⁺ retention can lead to increased muscle protein degradation and muscle wasting. In addition, through different synergistic pathways, H⁺ retention can also increase bone dissolution, cell-mediated bone resorption, and decrease bone formation. Similarly, H⁺ retention can also result in renal injury and nephrolithiasis, and may accelerate progression of CKD.

Pulmonary ventilation is controlled by partial arterial pressure of CO₂ (pCO₂), partial pressure of oxygen, and pH. Central chemoreceptors (located near the ventral surface of the medulla oblongata of the brain) and peripheral chemoreceptors (located in the carotid bodies and aortic bodies of the aortic arch) respond to changes in pCO₂ by triggering a respiratory response, which in turn affects bicarbonate (HCO₃ or HCO₃⁻) concentration and, thereby, changes in the pH level. Similarly, the kidney is responsible for the regulation of HCO₃ through reabsorption, production, and, in some situations, excretion of HCO₃. The kidneys reabsorb almost all of the altered HCO₃ in the proximal and distal tubular segments of the nephrons and produce new HCO₃ to replace the amount consumed by acids through excretion of titratable acids and ammonium.

Pure alterations in acid-base homeostasis may include one of four primary disorders: metabolic acidosis, metabolic alkalosis, respiratory acidosis, and respiratory alkalosis. In addition to these pure acid-base alterations, combinations can occur (“mixed” acid-base disorders). An acid-base disorder is metabolic or respiratory depending on whether the changes in HCO₃ or in pCO₂ are due to abnormalities of renal or respiratory functions, respectively. In particular, an acid-base disorder is termed metabolic when the primary abnormality can be attributed to changes in HCO_{3,} either as a result of an imbalance between net H⁺ production and renal HCO₃ reabsorption, or due to HCO₃ renal or gastrointestinal absorptive and secretion defects. An acid-base disorder may be termed respiratory if the primary abnormality is due to changes in pCO₂ caused by imbalances between metabolic production and pulmonary excretion of CO₂ or an abnormality in respiratory function. The status of acid-base disorders is acidotic or alkalotic if the blood pH is below or above the normal physiological range, respectively. In some embodiments, the normal physiological range may be a pH of about 7.40±0.02. Accordingly, acidosis or alkalosis may refer to the process in which H⁺ concentration is increased or decreased, respectively.

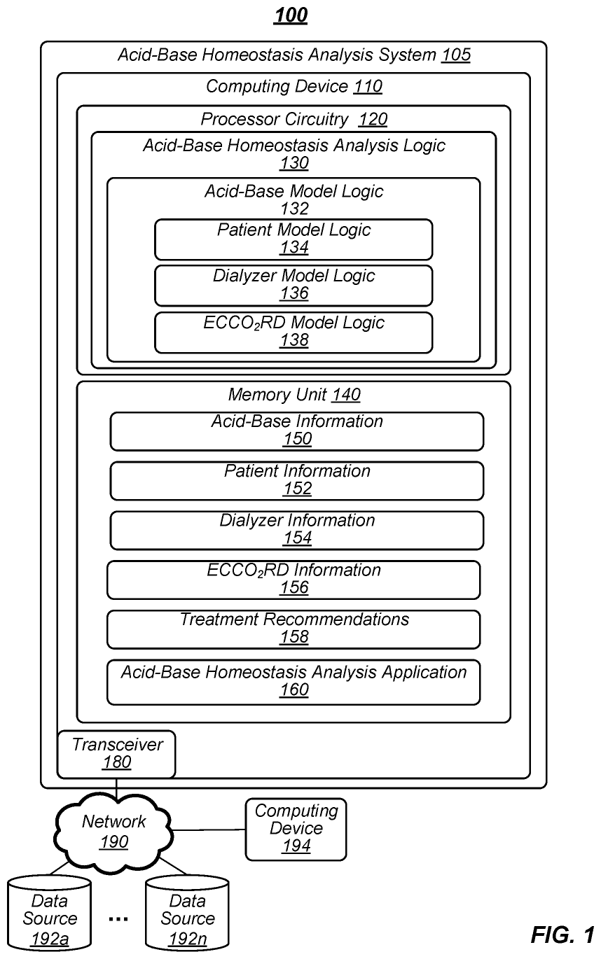

FIG. 1 illustrates an example of an operating environment 100 that may be representative of some embodiments. As shown in FIG. 1, operating environment 100 may include an acid-base homeostasis analysis system 105. In various embodiments, acid-base homeostasis analysis system 105 may include a computing device 110 communicatively coupled to network 190 via a transceiver 180. In some embodiments, computing device 110 may be a server computer or other type of computing device.

Computing device 110 may be configured to manage, among other things, operational aspects of an acid-base homeostasis process according to some embodiments. Although only one computing device 110 is depicted in FIG. 1, embodiments are not so limited. In various embodiments, the functions, operations, configurations, data storage functions, applications, logic, and/or the like described with respect to computing device 110 may be performed by and/or stored in one or more other computing devices (not shown), for example, coupled to computing device 110 via network 190 (for instance, one or more of client or peer devices 194). A single computing device 110 is depicted for illustrative purposes only to simplify the figure. Embodiments are not limited in this context.

Computing device 110 may include a processor circuitry 120 that may include and/or may access various logics for performing processes according to some embodiments. For instance, processor circuitry 120 may include and/or may access acid-base homeostasis analysis logic 130, acid-base model logic 132, dialysis patient model logic 134, dialyzer model logic 136, and/or ECCO₂RD model logic 138. Processing circuitry 120, acid-base model logic 132, dialysis patient model logic 134, dialyzer model logic 136, and/or ECCO₂RD model logic 138, and/or portions thereof may be implemented in hardware, software, or a combination thereof. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution, examples of which are provided by the exemplary computing architecture 2800. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, a control loop, a computational model or application, an AI model or application, an ML model or application, variations thereof, combinations of any of the foregoing, and/or the like.

Although acid-base homeostasis analysis logic 130 is depicted in FIG. 1 as being within processor circuitry 120, embodiments are not so limited. For example, acid-base model logic 132, dialysis patient model logic 134, dialyzer model logic 136, and/or ECCO₂RD model logic 138, and/or any component thereof may be located within an accelerator, a processor core, an interface, an individual processor die, implemented entirely as a software application (for instance, an acid-base homeostasis analysis application 160), and/or the like.

Memory unit 140 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (for example, USB memory, solid state drives (SSD) and any other type of storage media suitable for storing information. In addition, memory unit 140 may include various types of computer-readable storage media in the form of one or more lower speed memory units, including an internal (or external) hard disk drive (HDD), a magnetic floppy disk drive (FDD), and an optical disk drive to read from or write to a removable optical disk (for example, a CD-ROM or DVD), a solid state drive (SSD), and/or the like.

Memory unit 140 may store various types of information and/or applications for an acid-base homeostasis process according to some embodiments. For example, memory unit 140 may store acid-base information 150, patient information 152, dialyzer information 154, ECCO₂RD information 156, treatment recommendations 158, and/or acid-base homeostasis analysis application 160. In some embodiments, some or all of acid-base information 150, patient information 152, dialyzer information 154, ECCO₂RD information 156, treatment recommendations 158, and/or acid-base homeostasis analysis application 160 may be stored in one or more data stores 192a-n accessible to computing device 110 via network 190. For example, one or more of data stores 192a-n may be or may include a clinical data repository or database, a health information system (HIS), an electronic medical record (EMR) system, a dialysis information system (DIS), a picture archiving and communication system (PACS), a Centers for Medicare and Medicaid Services (CMS) database, U.S. Renal Data System (USRDS), a proprietary database, and/or the like. In some embodiments, memory 140 and/or data sources 192a-n may store historical patient population information, for example, used according to some embodiments to verify acid-base model outcomes.

In some embodiments, acid-base homeostasis analysis logic 130, for example, via acid-base model logic 132 and/or acid-base homeostasis analysis application 160, may operate to simulate acid-base homeostasis according to some embodiments. In various embodiments, acid-base homeostasis analysis logic 130, for example, via acid-base model logic 132 and/or acid-base homeostasis analysis application 160, may operate to simulate acid-base homeostasis for a dialysis patient undergoing dialysis treatment according to some embodiments. Dialysis patient model logic 134 may operate to implement a dialysis patient model according to various embodiments. Dialyzer model logic 1386 may operate to implement a dialyzer model according to some embodiments. ECCO₂RD model logic 138 may operate to implement an ECCO₂RD according to some embodiments.

In various embodiments, acid-base information 150 may include parameters, variables, values, and/or the like used by acid-base homeostasis analysis logic 130 and acid-base models implemented by acid-base homeostasis analysis logic 130 and/or components thereof. In various information, acid-base information 150 may include information generated by an acid-base model. For example, in various embodiments, acid-base information 150 may include predicted patient information determined by a model. For example, predicted patient information may include predicted pH (for instance, serum pH), pCO₂, and/or HCO₃.

In exemplary embodiments, patient information 152 may include information associated with patients and/or virtual patients modeled via acid-base models according to some embodiments and/or actual patients of historical information, for instance, used to teach or validate the acid-base models. Non-limiting examples of patient information 152 may include gender, age, weight, dry weight, treatment regimen (for instance, HD) and doses (for example, HCO₃), pre/post dialysis information (for example, pH, pCO₂, pO₂, HCO3), and/or the like. In various embodiments, dialyzer information 154 may include information associated with an actual or virtual (i.e., modelled) dialyzer used for an acid-base model and/or validation thereof, such as ultrafiltration volume (UFV), UF rate (UFR), dialyzer type, machine model, treatment mode (hemodialysis, hemodiafiltration, etc.), operating parameters, and/or the like. In various embodiments, ECCO₂RD 156 may include information associated with an actual or virtual ECCO₂RD and/or operation thereof

In some embodiments, treatment recommendations 158 may include treatment recommendations, suggestions, plans, information, and/or the like generated by an acid-base model according to various embodiments. For example, an acid-base model according to some embodiments may generate a treatment recommendation 158 for a real-world patient and/or patient population based on a model outcome generated for a corresponding virtual patient and/or patient population. Non-limiting examples of treatment recommendations may include administering HCO₃⁻ supplementation, acid-binders, hemodialysis bicarbonate dialysate, and/or patient diet instructions (for instance, restricting overconsumption of acidogenic diets). In a particular example, a treatment recommendation 158 may include a prescription of an optimized dialysate bicarbonate concentration to restore acid-base homeostasis without generating an “overshoot” metabolic alkalosis. In some embodiments, a treatment recommendation 158 for a patient and/or patient population (for instance, population of patients with particular characteristics (e.g., gender, weight, health condition, and/or the like)) may be generated based on a primary parameter and/or predicted patient information for patients and/or patient populations (and/or virtual implementations thereof) associated with the patient and/or patient population. In some embodiments, a primary parameter may include one or more parameters that influence a condition. For example, for healthy individuals, the predominant parameters affecting pH are those involving renal function (acid secretion rate (ϕ_{CO}_{₂}) and HCO₃⁻ reabsorption rate (D_{HCO}_{₃}_{^{−}})), HCO₃⁻ therapy (J_{HCO}_{₃}_{^{−}}), reaction rates or pKₐ of the buffer system, production (P_{H}) and removal or non-bicarbonate buffering of protons (γ_{H}). Therapies targeting these parameters may have a strong effect on correcting pH disturbances. Accordingly, these may be primary parameters for pH acid-base homeostasis for healthy individuals. For individuals with metabolic acidosis, primary parameters may include respiratory CO₂ removal (D_{CO}_{₂}), HCO₃⁻ supplementation or therapy (P_{HCO}_{₃}_{^{−}}, for example, NaHCO₃ or HD), hydration reaction rate (K_{H}_{⁺}_{, HCO}_{₃}_{^{−}}), and/or removal of excess protons (for example, through acid-binder supplementation) will be effective.

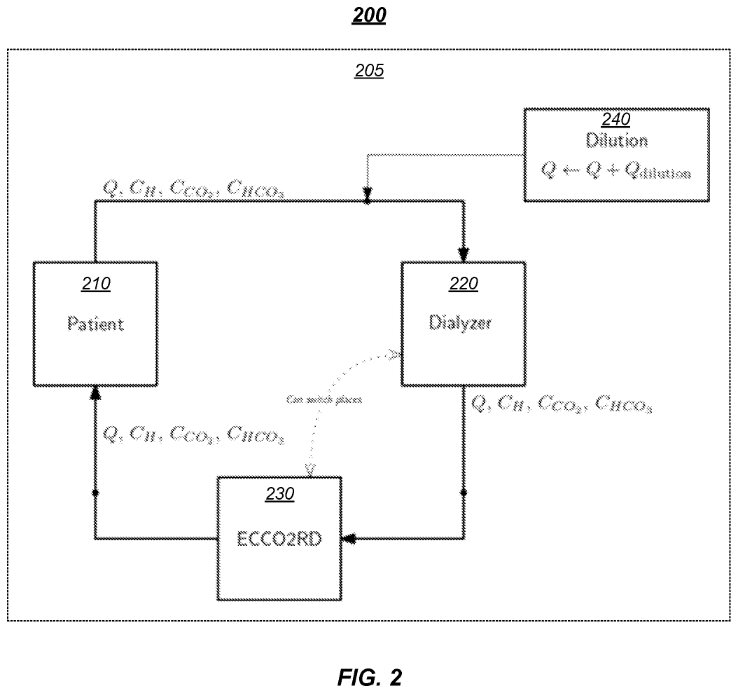

In some embodiments, acid-base models, such as implemented via acid-base model logic 132 (see, for example, FIG. 2) may operate to model acid-basis homeostasis for a patient and/or population of patients to determine predicted patient information (e.g., pH level, pCO₂ level, and/or HCO₃ level). In various embodiments, acid-base models may model an acid-base disorder for a certain patient population to determine predicted patient information (e.g., pH level, pCO₂ level, and/or HCO₃ level). The acid-base models may be used to determine predicted patient information for certain treatments (predicted treatment information), which may model treatment results for a patient. In some embodiments, a treatment recommendation may include maintaining/regulating acid-base homeostasis. Due to the accuracy of the acid-base models described according to some embodiments in predicting treatment outcomes, the acid-base models may be used to generate useful treatment recommendations for patients compared with conventional systems. In some embodiments, primary parameters may be determined based on predicted patient information (for instance, running an acid-base model to determine primary parameters affecting an acid-base disorder). Treatment recommendations 158 may be targeted to affect primary parameters in order to effectively address a condition. Embodiments are not limited in this context.

Some embodiments may provide an acid-base model in the form of a physiological acid-base model. In various embodiments, a physiological acid-base model may provide a physiologically-based model describing acid-base homeostasis under normal (or substantially normal) physiologic conditions that may be used, for example, to analyze the effects of pathophysiologic acid-base perturbations on the acid-base status of a patient (or virtual patient). In exemplary embodiments, a physiological acid-base model may operate to model, process, analyze, experiment, or otherwise simulate, among other things, the physiological regulation of HCO₃⁻/CO₂ buffering system with Henderson-Hasselbalch mass-action kinetics, endogenous production of both CO₂ and H⁺, non-bicarbonate buffering, and/or renal and respiratory regulations.

Various embodiments may provide acid-base models in the form of intradialytic models. In some embodiments, the intradialytic models may be or may include a dialysis patient model (for instance, implemented via dialysis patient model logic 134) that may include an implementation of a physiological acid-base model for a dialysis patient, for example, characterized by impaired renal regulation that has been replaced by dialysis (for instance, hemodialysis (HD)).

As described in more detail below, the acid-base models according to some embodiments may be used to predict treatment outcomes and/or to provide treatment recommendations for various acid-base disorders. In some embodiments, certain model parameters may be altered to determine their effect on acid-base homeostasis. For instance, changes to an acid secretion rate parameter and a renal filtration rate parameter (for example, reducing or eliminating these parameters (e.g., setting their model value to zero)) may simulate an acid-base disorder, such as renal failure, metabolic acidosis, metabolic alkalosis, respiratory acidosis, respiratory alkalosis, and/or the like. Based on the particular configuration and operating processes of acid-base models according to some embodiments, they may be used to accurately predict changes to parameters affecting acid-base homeostasis.

In some embodiments, acid-base homeostasis logic 130 may operate to receive patient information 152 for a particular patient (for instance, gender, age, health (for example, renal failure, normal, and/or the like), HCO₃ level, pH, and/or the like) and determine a treatment recommendation 156 for maintaining acid-base homeostasis and/or treating an acid-base disorder. For example, in a healthy person, correcting a deficient HCO₃ level may be different than for a patient experiencing metabolic acidosis. In some embodiments, acid-base models may be used to model, predict, or otherwise process various treatment models (for instance, acid-binder therapies, HCO₃ therapies, and/or the like) to determine, predict, or otherwise analyze treatment outcomes that may be used to determine actual patient therapy regimens. In some embodiments, acid-base homeostasis logic 130 may include, implement, or otherwise process a feedback loop (or iterative) function for ongoing use of the acid-base models to adjust acid-base conditions, including through dialysis modifications (for example, adjusting ultrafiltration rate and/or volume), or administration of drugs, bicarbonate, etc. based on continuous predicted patient information. In some embodiments, as a treatment is administered (for example, during dialysis, supplement dosages, and/or the like), the treatment information may be used as input into an acid-base model to generate updated predicted patient information, which may then be used to determine treatment results and/or to update a treatment recommendation (including in real or substantially real time, for example, during a dialysis treatment).

FIG. 2 illustrates an example of an operating environment 200 that may be representative of some embodiments. As shown in FIG. 2, operating environment 200 may include an acid-base model 205 depicting an illustrative model structure in accordance with the present disclosure. For example, FIG. 2 may represent the structure between different elements of an acid-base model according to some embodiments. In various embodiments, acid-base model 205 may include a patient model (or element) 210, a dialyzer model 220, an ECCO₂RD model 230, and/or a dilution model 240.

The configuration of acid-base model 205 depicted in FIG. 2 is for non-limiting illustrative purposes. For example, acid-base model 205 may have different forms, configurations, and/or the like according to some embodiments. For instance, acid-base model 205 may have more or less elements, and/or elements arranged in different configurations. For example, acid-base model may not include dialyzer model 220 and/or ECCO₂RD model 230. In another example, dialyzer model 220 and/or ECCO₂RD model 230 may be in different positions (e.g., as indicated in FIG. 2, dialyzer model 220 and ECCO₂RD model 230 may have their positions switched).

In a further example, dilution model 240 may be inserted anywhere in the circuit (e.g., between patient model 210 and dialyzer model 220; between dialyzer model 220 and ECCO₂RD model 230; between ECCO₂RD model 230 and patient model 210; and/or the like). In various embodiments, dilution model 240 may operate or be represented by the increase of the blood flow rate (Q) in between models 210, 220, and/or 230, and the appropriate decrease in concentrations.

In some embodiments, models 210, 220, 230, and/or 240 of acid-base model 205 may exchange values of Q and of concentrations of H⁺, HCO⁻ and CO₂ (e.g., C_{H}, C_{HCO}, C_{CO}). These input/outputs may be represented as boundary conditions within acid-base model 205.

Concentrations of hydrogen and CO₂ may be expressed in terms of the solution pH and carbon dioxide partial pressure (pCO₂). In some embodiments, the conversion between pH and may be made according to the following Equation (1):

$$pH = - {log}_{10} {C}_{{H}^{+}} \\ {pCO}_{2} = \frac{{C}_{{CO}_{2}}}{{k}_{{CO}_{2}}} , \qquad ( 1 )$$

where k_{CO}_{₂}=3.0×10⁻⁸ M/mmHg.

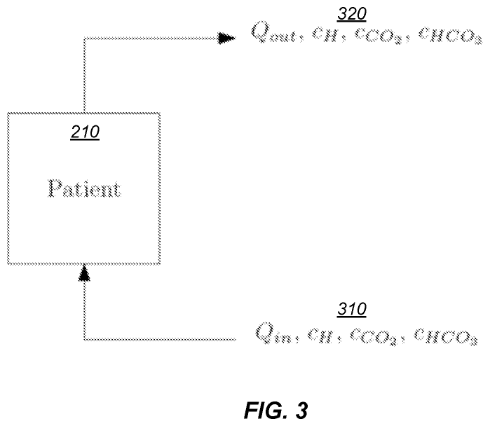

FIG. 3 illustrates an example of a patient model 210 that may be representative of some embodiments. In general, FIG. 3 depicts input 310 and output 320 from a patient (or patient model) 210 perspective. In various embodiments, input 310 may be or may include Qᵢₙ, C_{H}, c_{CO}_{₂}, c_{HCO}_{₃}. In some embodiments, output 320 may be or may include Qₒᵤₜ, C_{H}, c_{CO}_{₂}, c_{HCO}_{₃}.

In some embodiments, the homeostatic dynamics of the HCO₃_{^{−}}/CO₂ acid-base system may be the same or similar to dynamics described in Cherif et al. In some embodiments, equations that describe the homeostatic dynamics of the HCO₃_{^{−}}/CO₂ acid-base system may include the following Equations (2)−(4):

$$\frac{{dC}_{H}}{dt} = {P}_{H} - γ {C}_{H} + \qquad ( 2 )$$

$$\frac{{dC}_{CO 2}}{dt} = {P}_{CO 2} - {D}_{CO 2} {V}_{0} {C}_{CO 2} - \qquad ( 3 )$$

$$\frac{{dC}_{HCO 3}}{dt} = {P}_{HCO 3} - {ϕ}_{{CO}_{2}} {C}_{{CO}_{{2}_{0}}} - {D}_{HCO 3} {C}_{HCO 3} + , \qquad ( 4 )$$

where [symbol] =K_{H,HCO3} C_{H}C_{HCO3}+K_{CO2} C_{CO2} comes from the Henderson-Hasselbach mass-action kinetics.

When patient model 210 is included in a blood circuit with a dialyzer (for instance, dialyzer model 220), changes in total blood volume (for example, through fluid removal) may be included. From a patient perspective, such as via patient model 210 such a change may be given by the following Equation (5):

$$\frac{dV}{dt} = {Q}_{in} - {Q}_{out} \qquad ( 5 )$$

In various embodiments, Equations (2)-(4) may be re-written in terms of the number of moles, such as, for each concentration Cᵢ=nᵢ/V, the derivative for nᵢ, may be given by the following Equation (6):

$$\frac{{dn}_{i}}{dt} = \frac{{dC}_{i}}{dt} V + \frac{dV}{dt} {C}_{i} = \frac{{dC}_{i}}{dt} V + {Q}_{in} {C}_{i , in} - {Q}_{out} {C}_{i} \qquad ( 6 )$$

In some embodiments, where it is included, the concentration of the incoming flow rate (C_{i,in}) may not be the same as the concentration of the outgoing flow; accordingly, the acid-base model 205 may include the following Equation (7) (or Equations (7a)-(7d)):

$$\frac{{dn}_{H}}{dt} = {Q}_{in} {C}_{H , in} - {Q}_{out} {C}_{H} + ( {P}_{H} - γ {C}_{H} + ) V \qquad ( 7 a )$$

$$\frac{{dn}_{{CO}_{2}}}{dt} = {Q}_{in} {C}_{{CO}_{2} , in} - {Q}_{out} {C}_{{CO}_{2}} + ( {P}_{{CO}_{2}} - {D}_{CO 2} {𝒱}_{0} {C}_{{CO}_{2}} - ) V \qquad ( 7 b )$$

$$\frac{{dn}_{{HCO}_{3}}}{dt} = {Q}_{in} {C}_{{HCO}_{3} , in} - {Q}_{out} {C}_{{HCO}_{3}} + ( {P}_{{HCO}_{3}} + {ρ}_{residual} ( {ϕ}_{{CO}_{2}} {C}_{{CO}_{2}} - {D}_{{HCO}_{3}} {C}_{{HCO}_{3}} ) + ) V \qquad ( 7 c )$$

$$\frac{dV}{dt} = {Q}_{in} - {Q}_{out} \qquad ( 7 d )$$

In various embodiments in which each concentration may be calculated as Cᵢ=nᵢ/V, if Qᵢₙ=Qₒᵤₜ=0, then \dot{V}=0⇒V=constant, then Equations (7a)-(7d) can be rewritten back to their original form of Equations (2)-(4).

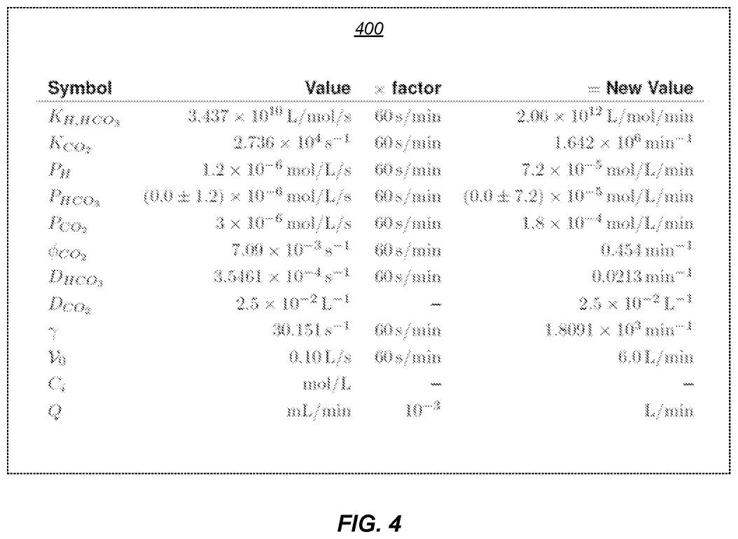

FIG. 4 illustrates an example Table 400 in accordance with the present disclosure. More specifically, Table 400 represents the values and units of the constants and variables in Cherif et al., as well as their respective values in the SI units. When applicable, a conversion factor is also present. The values in Table 400 were adjusted, for instance, to cause the point pH=7.4, pCO₂=40 mmHg, and C_{HCO3}=2.4 mM to be fixed points of Equations (7a)-7(d).

In some embodiments, dialyzer model 220 may be configured the same or very similar to a parallel plates heat exchanger or model thereof. In various embodiments, the equations for a dialyzer model may be the same as, similar to, based on, and/or adapted from equations described in the Acid-Base Dynamics Disclosure.

In various embodiments, dialyzer model 220 may be configured to simulate intradialytic dynamics associated with pH and acid-base homeostasis. In some embodiments, dialyzer model 220 may be configured to model an HD dialyzer. In exemplary embodiments, dialyzer model 220 may be configured for quantitating the intradialytic dynamics of HCO3− and H+, which may be parameterize to model anuric patients receiving HD. In some embodiments, dialyzer model 220 may include, may be the same or substantially the same as, or may be an adaptation of the dialyzer model described in Maheshwari et al., “An In Silico Method to Predict Net Calcium Transfer During Hemodialysis,” 2017 39th Annual International Conference of the IEEE Engineering in Medicine and Biology Society (EMBC), pp. 2740-2743 (2017).

In various embodiments, acid-base model 205 and/or models thereof, may be the same or similar to a physiological acid-base model, for example, as described in the Acid-Base Dynamics Disclosure.

For example, a physiological acid-base model according to some embodiments may operate to model the effect of systemic acid-base homeostasis. In some embodiments, a physiological acid-base model may be implemented using a system of coupled nonlinear ordinary differential equations, for example, to describe the acid-base buffering kinetics through the HCO₃-CO₂ system and incorporating the relevant physiological regulatory mechanisms. In some embodiments, physiological acid-base models may focus on HCO₃-CO₂ buffering kinetics, which is the most effective buffer system that controls systemic pH.

The pH of extracellular fluid is mainly regulated by the following three mechanisms, which act on different timescales: (i) chemical acid-base buffering, (ii) respiratory control, and (iii) renal filtration. The following Equation (8) provides an illustrative and non-restrictive HCO3 buffering system according to some embodiments:

$${HCO}_{3}^{-} + {H}^{+} {⇌}_{{K}_{{CO}_{2}}}^{{K}_{{H}^{+} , {HCO}_{3}^{-}}} {CO}_{2} + {H}_{2} O . \qquad ( 8 )$$

In some embodiments, in Equation (8), it may be assumed that carbonic anhydrase (CA) accelerates the carbonic acid reactions.

Chemical acid-base buffering prevents excessive changes in pH, where the timescale of this process is usually in seconds. The ability of the lung to increase or decrease ventilation allows it to regulate CO₂ removal as a gas in the expired air from the extracellular fluid, thereby adjusting the pH. In particular, due to continuous production of CO₂ as a by-product of cellular metabolism, the ventilation rate must be able to accommodate alterations in CO₂ in order to equilibrate the pH of the extracellular fluid. Although the process is fast, for example, occurring in a matter of minutes, it is less effective than chemical buffering. If the acid-base imbalance persists, then kidneys excrete either excess acid or base, an adaptation process that takes hours to days.

The kidneys, representing a very powerful regulatory system, have the ability to secrete large amounts of H⁺ into the tubular lumen during metabolic acidosis. Also, excretion and reabsorption of HCO₃ take place in the proximal tubule and distal tubule. In particular, in the proximal tubule, H⁺ secreted through a Na⁺/H⁺ countertransport-facilitated process, while HCO₃ is reabsorbed by combining with H⁺ to form carbonic acid(H₂CO₃) which is converted into CO₂ and H₂O (via carbonic anhydrase enzymatic activity). In the intercalated cells, H⁺/Cl⁻cotransporter facilitates the secretion of H⁺. Although this process only accounts for 5% of the secreted Et, it provides a gradient for the further secretion of more H⁺ into the tubule lumen. Most of the secreted H⁺ is used to reclaim the filtered HCO_{3,} and this rate of HCO₃ reabsorption is related to the rate of acid excretion.

To model the acid-base homeostatic process, physiological acid-base models according to some embodiments may track the concentrations of bicarbonate (Y_{HCO}_{₃}_{^{−}}), carbon dioxide (Y_{CO}_{₂}) and free hydrogen protons (Y_{H}_{⁺}). Using Henderson-Hasselbalch mass-action kinetics with renal and pulmonary regulatory mechanisms, the homeostatic dynamics of the HCO₃/CO₂ acid-base system, as modelled by physiological acid-base models according to some embodiments may be configured according to the following Equations (9)-(11) (i.e., the physiological acid-base model equations or bicarbonate buffer kinetic system):

$$\frac{{dY}_{{H}^{+}}}{dt} = {P}_{{H}^{+}} - {γ}_{{H}^{+}} {Y}_{{H}^{+}} - {K}_{{H}^{+} , {HCO}_{3}} - {Y}_{{H}^{+}} {Y}_{{HCO}_{3}^{-}} + {K}_{{CO}_{2}} {Y}_{{CO}_{2}} , \qquad ( 9 )$$

$$\frac{{dY}_{{HCO}_{3}^{-}}}{dt} = {J}_{{HCO}_{3}^{-}} + {ϕ}_{{CO}_{2}} {Y}_{{CO}_{2}} - {D}_{{HCO}_{3}^{-}} {Y}_{{HCO}_{3}^{-}} - {K}_{{H}^{+} , {HCO}_{3}^{-}} {Y}_{{H}^{+}} {Y}_{{HCO}_{3}^{-}} + {K}_{{CO}_{2}} {Y}_{{CO}_{2}} , \qquad ( 10 )$$

$$\frac{{dY}_{{CO}_{2}}}{dt} = {P}_{{CO}_{2}} - {D}_{{CO}_{2}} {𝒱}_{0} {Y}_{{CO}_{2}} + {K}_{{H}^{+} , {HCO}_{3}} - {Y}_{{H}^{+}} {Y}_{{HCO}_{3}^{-}} - {K}_{{CO}_{2}} {Y}_{{CO}_{2}} , \qquad ( 11 )$$

In some embodiments, in Equations (9)-(11) tϵ [symbol] ₀:=[0, +∞) the following initial conditions: Y_{H}_{⁺}(0)=H₀, Y_{HCO}_{₃}_{^{−}}(0)=B₀, and Y_{CO}_{₂}(0)=C₀, may set a patient (or virtual patient) to a normal physiological state. The parameter P_{H}_{⁺} represents the cellular production of H⁺, γ_{H}_{⁺}, denotes H⁺ loss either due to renal clearance and/or non-bicarbonate buffering (for example, buffering with albumin, Ca²⁺, PO₄³⁻). The hydration and de-hydration reaction rates are given by the parameters K_{H}_{⁺}_{,HCO}_{₃}_{^{−}} and K_{CO}_{₂}, respectively, where the values may be adjusted to reflect the carbonic anhydrase activity. In addition, J_{HCO}_{₃}_{^{−}} denotes HCO₃⁻ therapy and/or supplementation, ϕ_{CO}_{₂}, represents the acid secretion rate, P_{CO}_{₂},is the body or cellular (mitochondrial) production of CO₂, and D_{HCO}_{₃}_{^{−}} represents the renal filtration rate of HCO₃⁻. The effective ventilation rate (D_{CO}_{₂}V₀) captures the pulmonary removal of CO₂, where V₀ is the minute volume ventilation, and D_{CO}_{₂} is the ventilation rate. In some embodiments, the kinetics of H₂O is not included in Equations (9)-(11) because H₂O is assumed to be abundant as a solvent. In some embodiments, Equations (9)-(11) may be formulated via general expressions (see, for example, Equations (12) and (13)), for instance, with non-linear ventilation (D_{CO}_{₂}V₀Y_{CO}_{₂})).

In various embodiments, the first two terms in Equation (9) may account for production of P_{H}_{⁺}(t) of H⁺ from the body, either through consumption and/or through other processes (for example, cellular metabolism), and for removal of H⁺ as either a titratable acid and/or ammonium or non-carbonate buffering (for instance, buffering with phosphate and/or calcium). These two terms collectively correspond to H⁺ mobilization due to buffering with non-bicarbonate buffers and other processes. The third and last terms in Equation (9) correspond to buffering reaction kinetics.

For Equation (10), the first term, J_{HCO}_{₃}_{^{−}}, represents HCO₃⁻ therapy, the second and third terms, ϕ_{CO}_{₂}Y_{CO}_{₂}−D_{HCO}_{₃}_{^{−Y}}_{HCO}_{₃}_{^{−}}, may describe renal filtration processes, where it is assumed that the amount of HCO₃⁻ lost to kidney from the blood through filtration may be related to filtered load, and the equivalence of HCO₃⁻ reabsorption and acid excretion is through the splitting of CO₂ by intracellular carbonic anhydrase enzymatic activity. For example, an increase in CO₂ concentration may increase the conversion of CO₂ into H⁺ and HCO₃⁻ in a normally functioning kidney, which may result in higher acid secretion into the urine and CO₂ absorption into the bloodstream. Accordingly, the second term of Equation (3) may describe acid secretion, whereas the third term is the HCO₃⁻ load to be filtered.

Similarly, Equation (11) may be directed to buffering kinetics, where the first term, P_{CO}_{₂}, may be production of CO₂ in the body (for example, a cellular metabolic or mitochondrial process), and the second term, D_{CO}_{₂}V₀Y_{CO}_{₂}, is the removal of CO₂ through respiratory ventilation by the lung, which may depend on blood volume, cardiac output, arteriovenous difference (for example, the concentration difference between arterial and venous blood) of CO₂. To simplify the model, some embodiments may assume that ventilation rate V₀ is constant; however, V₀ may depend on pCO₂, oxygen partial pressure (pO₂), and/or pH.

In some embodiments, the term D_{CO}_{₂}V₀Y_{CO}_{₂}, may be nonlinear and may represent the effective ventilation rate, D_{CO2}V(Y_{CO2})Y_{CO}_{₂}, where D^{CO2} becomes the ventilation rate and V denotes the minute volume ventilation. The empirical ventilation function may take the form of one of the following Equations (12) or (13):

V₁(pCO₂,pO₂)=V₀+Vₚe^{-0.05}^{ₚ}^{O2(t)} max{0,pCO₂(t)−Iₚ} (12),

V₂(pCO₂,pO₂)=V₀+Vₚ(102.4−S_{O}_{₂}(t))max{0,pCO₂(t)−Iₚ} (13),

where V₀ and Vₚ represent baseline and slope parameters, and Iₚ is the cutoff threshold. In the ventilation term, the role of pH sensors in the medulla oblongata in regulating bicarbonate buffering homeostasis is disregarded as de minimus. The ventilation term V₁(pCO₂,pO₂) above relates the interaction between partial pressure pCO₂ and pO₂ and their effects on ventilation. In V₂(pCO₂,pO₂),V₂(pCO₂,pO₂) uses an oxygen saturation function, So, which may be expressed in terms of pO₂. Y_{CO}_{₂}, and pCO₂ are related by Y_{CO}_{₂}=0.03[(mmol/L)/mmHg]×pCO₂. In some embodiments, we may also assume that pO₂ is constant. At normal pO₂, ventilation increases by 2.5 L/min for every 1 mm Hg increase of pCO₂. From the above Equations (5) and (6) and FIG. 2, lowering pia increases ventilation for a given pCO₂ and the steepness of the net effective slope of ventilation. The term Vo can be replaced by either of the functional expressions above under the assumption that pia is constant or exogenously provided. In some embodiments, the simplified version may be used to determine acid-base dynamics according to some embodiments, thereby reducing the number of model parameters that need to be identified. In other embodiments, all the effects of ventilation may be lumped into ventilatory rate parameters. In some embodiments, Equations (12) and (13) may provide general physiological acid-base models (for example, with Equations (9)-(11) being a specific implementation thereof).

In various embodiments, acid-base model 205 and/or models thereof, may be the same or similar to an intradialytic acid-base model, for example, as described in the Acid-Base Dynamics Disclosure.

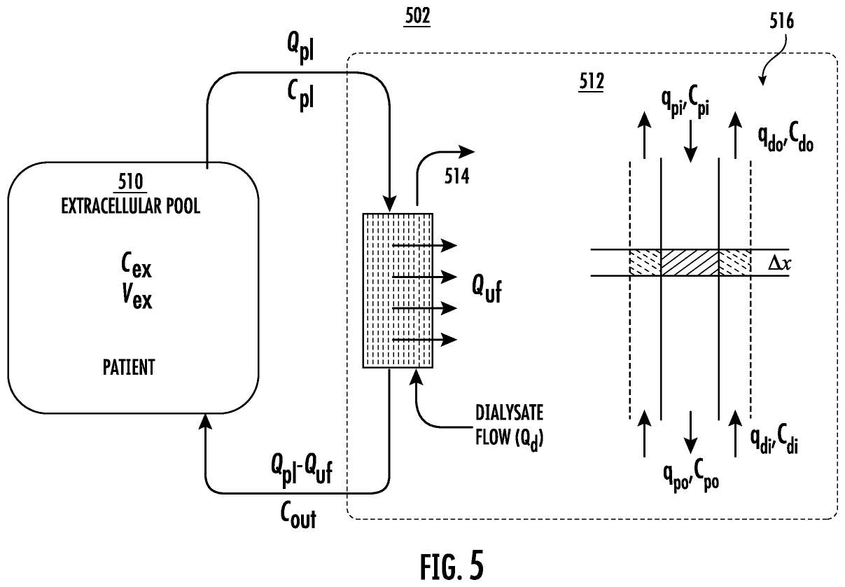

FIG. 5 illustrates a block diagram of an intradialytic acid-base model according to some embodiments. As shown in FIG. 5, an intradialytic acid-base model 502 may include a dialysis patient model or compartment 510 and a dialyzer model or compartment 512 having a dialyzer 514. In various embodiments, patient compartment 510 may include the distribution volume (Vₑₓ) and concentrations of acid-base variables (for example, Cₑₓ={C_{H}_{⁺},C_{HCO}_{₃}_{^{−}},C_{CO}_{₂}}). In some embodiments, Qₚ, Q_{d}, and Q_{uf} are the plasma flow rate, dialysate flow rate, and ultrafiltration rate, respectively. A single dialyzer fiber 516 may depict the counter-current flows interaction between blood and dialysate flows in dialyzer 514, in which there is an intradialytic transfer between blood and dialysate through the infinitesimal fiber segment Δx. The subscripts for q and C denote plasma (pl) or extracellular fluid (ex), dialyzer input (di) and output (do), dialyzer blood inlet (pi), and outlet (out).

For the dialysis patient model, some embodiments may use a physiologically-based dynamic model describing the regulation of HCO₃⁻/CO₂ buffering system with Henderson-Hasselbalch mass-action kinetics, in which we incorporate the endogenous production of both CO₂ and H⁺, non-bicarbonate buffering, and the physiologic regulation of the HCO₃⁻/CO₂ buffering system through ventilation and renal excretion. Although there are other buffer systems, some embodiments may focus mainly on the HCO₃⁻/CO₂ buffering system, which is the most abundance and effective buffer system in the body. Under physiologic conditions, pH is regulated mainly by chemical acid-base buffering, respiratory control and renal glomerular filtration. The chemical acid-base buffering is modeled using Henderson-Hasselbalch mass-action kinetics, in which, in some embodiments, the action of carbonic anhydrase is fast, and K_{H}_{⁺}_{,HCO}_{₃} _{^{−}} and K_{CO}_{₂} association and disassociate rates, respectively.

During normal cellular metabolic activities, CO₂ and H⁺ are produced as by-products; this endogenous production is captured by P_{CO}_{₂} and P_{H}_{⁺} for CO₂ and H⁺, respectively. In addition, acid is mobilized or buffered with other non-bicarbonate buffers such as phosphate, and this is represented by γ_{H}_{⁺}C_{H}_{⁺}, where γ_{H}_{⁺} is the mobilization or removal rate. In normal physiologic conditions, the kidney, the second regulatory organ, is responsible for regulating HCO₃_{^{−}} level by either secreting excess H+ into tubular lumen during metabolic acidosis, and/or excreting or reabsorbing HCO₃_{^{−}} in the proximal and distal segments of nephrons. Unlike the physiological acid-base model according to some embodiments, the dialysis patient model may not include (or may include lower functioning of) the renal regulation of HCO₃_{^{−}} and H⁺ because the dialysis patient model is adapted to dialysis patients where the ability of excrete acid is impaired. As a result, in some embodiments, the dialysis patient model may assume that there is little or even no renal function. In some embodiments, the renal function is replaced by the use of the dialyzer (for example, by dialyzer fluxes) implemented via the dialyzer model. For example, the expressions −QₚC_{HCO}_{₃}_{^{−}} and (Qₚ−Q_{uf})C_{HCO}_{₃}_{^{−}}_{,out} may account for bicarb flux from patient and post-dialyzer flux to patient, respectively, which is characterized by blood flow rate, Qₚ, and ultrafiltrate rate, Q_{uf}. For the third regulatory mechanism, in order to regulate CO₂ removal, lung increases or decreases ventilation, which is triggered by the response of central and peripheral chemoreceptors to changes in pCO₂, and D_{CO}_{₂}V₀Y_{CO}_{₂} describes removal of CO₂ through ventilation by lung characterized by blood volume, cardiac output, arteriovenous difference of CO₂. The parameters D_{CO}_{₂},and V₀ are ventilation rate and minute ventilation, respectively. In some embodiments, the dialysis patient model describing acid-base homeostasis may be according to the following Equations (14)-(16):

$$\frac{d ( {C}_{{H}^{+}} {V}_{ex} )}{dt} = {\underbrace{{P}_{{H}^{+}}}}_{Endogenous {H}^{+} production} - {\underbrace{{γ}_{{H}^{+}} {C}_{{H}^{+}}}}_{Lumped non - bicarbonate} - {\underbrace{( {K}_{{H}^{+} , {HCO}_{3}} - {C}_{{H}^{+}} {C}_{{HCO}_{3}^{-}} + {K}_{{CO}_{2}} {C}_{{CO}_{2}} ) {V}_{ex}}}_{Henderson - Hasselbach kinetics} , \qquad ( 14 )$$

$$\frac{d ( {C}_{{HCO}_{3}} - {V}_{ex} )}{dt} = {\underbrace{- {Q}_{p} {C}_{{HCO}_{3}^{-}}}}_{Flux from patient} + {\underbrace{( {Q}_{p} - {Q}_{uf} ) {C}_{{HCO}_{3}^{-} , out}}}_{Post - dialyzer flux to patient} - {\underbrace{( {K}_{{H}^{+} , {HCO}_{3}^{-}} {C}_{{H}^{+}} {C}_{{HCO}_{3}^{-}} + {K}_{{CO}_{2}} {C}_{{CO}_{2}} ) {V}_{ex}}}_{Henderson - Hasselbach kinetics} , \qquad ( 15 )$$

$$\frac{d ( {C}_{{CO}_{2}} {V}_{ex} )}{dt} = {\underbrace{{P}_{{CO}_{2}}}}_{Endogenous {CO}_{2} production} - {\underbrace{{D}_{{CO}_{2}} {V}_{0} {C}_{{CO}_{2}}}}_{Respiratory ventilation} + {\underbrace{( {K}_{{H}^{+} , {HCO}_{3}} - {C}_{{H}^{+}} {C}_{{HCO}_{3}^{-}} + {K}_{{CO}_{2}} {C}_{{CO}_{2}} ) {V}_{ex}}}_{Henderson - Hasselbach kinetics} . \qquad ( 16 )$$

During dialysis, a patient loses a significant amount of fluid. This is assumed to occur at a constant ultrafiltration rate, Q_{uf}, and fluid removal by ultrafiltration occurs in proportion to the compartmental distribution volume. Accordingly, in some embodiments, extracellular fluid volume may be determined according to the following Equation (17):

$$\frac{{dV}_{ex}}{dt} = {\underbrace{- {Q}_{uf}}}_{Ultrafiltration rate} . \qquad ( 17 )$$

In some embodiments, the dialyzer model may include two spatial temporal models describing both blood and dialysate sides using hyperbolic partial differential equations. The concentration of HCO₃_{^{−}} in the blood side may be determined according to the following Equation (18):

$$\frac{\partial {c}_{{HCO}_{3}^{-}}}{\partial t} = - {\underbrace{\frac{1}{N \cdot A} \frac{\partial ( {Q}_{p} {c}_{{HCO}_{3}^{-}} )}{\partial x}}}_{Axial convection} + {\underbrace{\frac{1}{N \cdot A} \frac{\partial {Q}_{p}}{\partial x} {c}_{{HCO}_{3}^{-}} ( 1 - {σ}_{{c}_{{HCO}_{3}^{-}}} )}}_{Radial convection} - {\underbrace{\frac{Pe}{{e}^{Pe} - 1} \frac{1}{N \cdot A \cdot L} {K}_{o} A ( {c}_{{HCO}_{3}^{-}} - \frac{{c}_{D , {HCO}_{3}^{-}}}{β} )}}_{Gibbs - Donnan corrected effective diffusion} , \qquad ( 18 )$$

where N is the number of fibers, A denotes fiber cross-sectional area,

$${σ}_{{c}_{H C {O}_{3}^{-}}}$$

represents the HCO₃_{^{−}} reflection coefficient,

$$Pe = \frac{( 1 - {σ}_{{c}_{{HCO}_{3}^{-}}} ) {Q}_{uf}}{{K}_{o} A}$$

defines the Peclet, L is the fiber length, KₒA is the effective membrane mass-transfer coefficient for HCO₃_{^{−}}, β is Gibbs-Donnan correction factor (i.e., a Gibbs-Donnan corrected dialyzer model). In some embodiments, β and may be set to a constant value of 1.05 which corresponds to 5% of HCO₃_{^{−}}. In various embodiments, for the dialyzer side, it may be assumed that the dialysate flow is uniform and equally shared by the N fibers present in the dialyzer housing. The hyperbolic partial differential equation describing the concentration within annulus dialysate flow boundary may have the form of the following Equation (19):

$$\frac{\partial {c}_{D , {HCO}_{3}^{-}}}{\partial t} = {\underbrace{\frac{1}{N \cdot {A}_{d}} \frac{\partial ( {Q}_{d} {c}_{D , {HCO}_{3}^{-}} )}{\partial x}}}_{Axial convection} - {\underbrace{\frac{1}{N \cdot {A}_{d}} \frac{\partial {Q}_{d}}{\partial x} {c}_{{HCO}_{3}^{-}} ( 1 - {σ}_{{c}_{{HCO}_{3}^{-}}} )}}_{Radial convection} + {\underbrace{\frac{Pe}{{e}^{Pe} - 1} \frac{1}{N \cdot {A}_{d} \cdot L} {K}_{o} A ( {c}_{{HCO}_{3}^{-}} - \frac{{c}_{D , {HCO}_{3}^{-}}}{β} )}}_{Gibbs - Donnan corrected effective diffusion} , \qquad ( 19 )$$

where A_{d} circular cross-sectional area of annulus space for dialysate flow around a fiber. In various embodiments, plasma flow rate (Qₚ) may decrease along the fiber length in the dialyzer due to ultrafiltration, the decrease may be linearly along the fiber length. In some embodiments, the dialysate flow rate may increase by the amount of fluid removed by ultrafiltration from the blood side to the dialysate side, resulting in counter-current kinetics. The spatial aspects of plasma and dialysate flow rates may be determined according to the following equations (20) and (21):

$${Q}_{p} = {Q}_{pi} - \frac{x}{L} {Q}_{uf} , \qquad ( 20 )$$

$${Q}_{d} = {Q}_{di} + \frac{( L - x )}{L} {Q}_{uf} , \qquad ( 21 )$$

where Qₚᵢ and Q_{di} are initial plasma and dialysate flow rates.

In some embodiments, Equations (14)-(21) may constitute components of intradialytic acid-base models describing, simulating, predicting, or otherwise processing intradialytic acid-base dynamics.

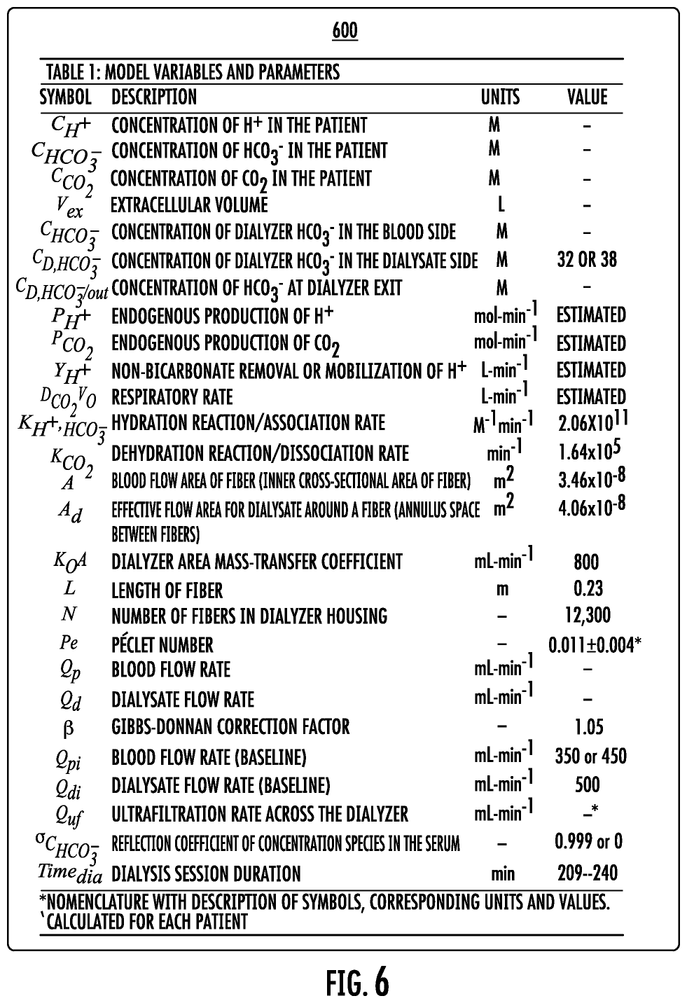

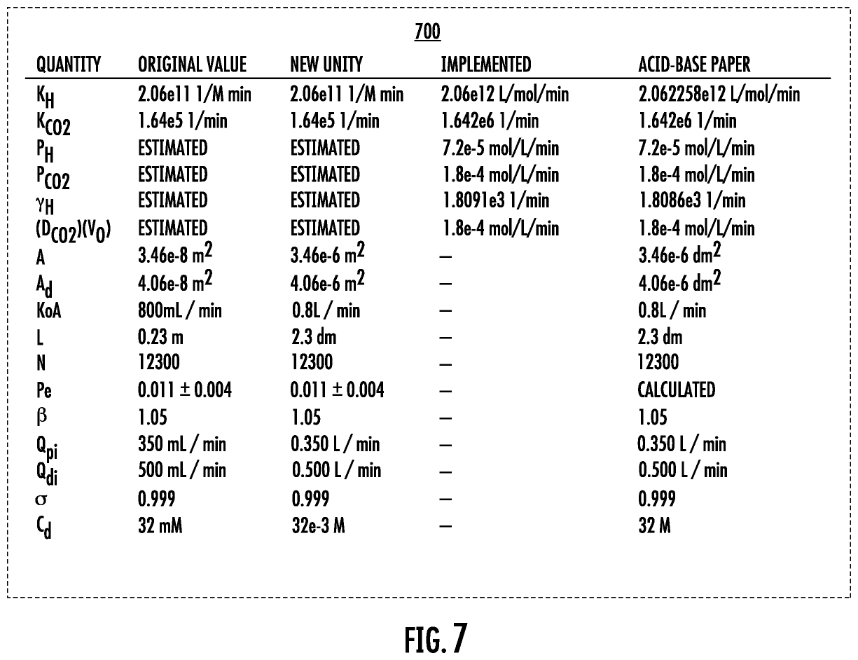

FIG. 6 illustrates table 600 of a first illustrative set of acid-base model parameters and values according to some embodiments. FIG. 7 illustrates table 700 of a second illustrative set of acid-base model parameters and values according to some embodiments. In some embodiments, for the dialyzer models, the difference between the values of the hydration and dehydration rates (K_{H} and K_{CO}_{₂}), may differ from one order of magnitude between those described in the Acid-Base Dynamics Disclosure.

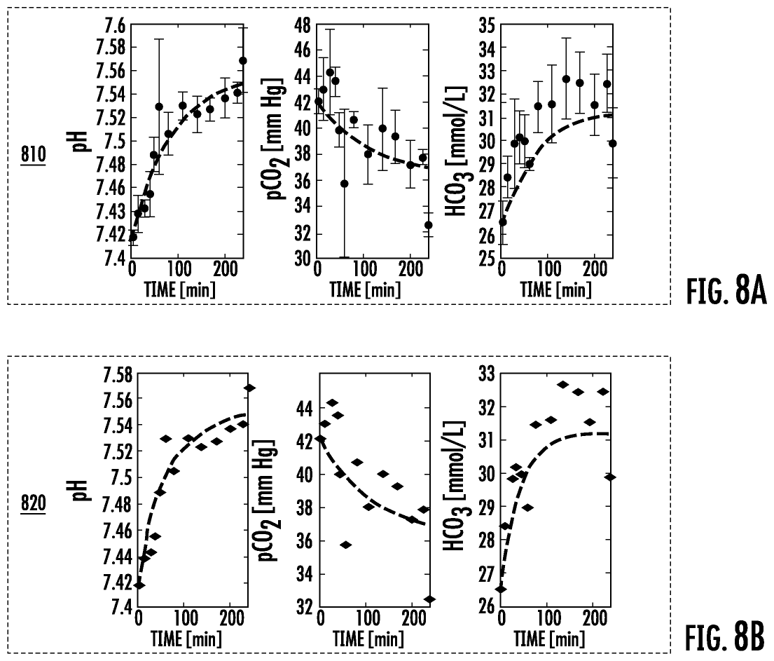

FIG. 8A and 8B illustrates results of a simulation of a physiological acid-base model according to some embodiments. More specifically, FIG. 8A depicts results 810 from acid-base models from the Acid-Base Dynamics Disclosure. FIG. 8B depicts results 820 from acid-base models according to some embodiments, for instance, acid-base model 205. The results in FIGS. 8A and 8B are obtained with only one ρ_{residual} being zero, for instance, when no residual kidney function is included in the model.

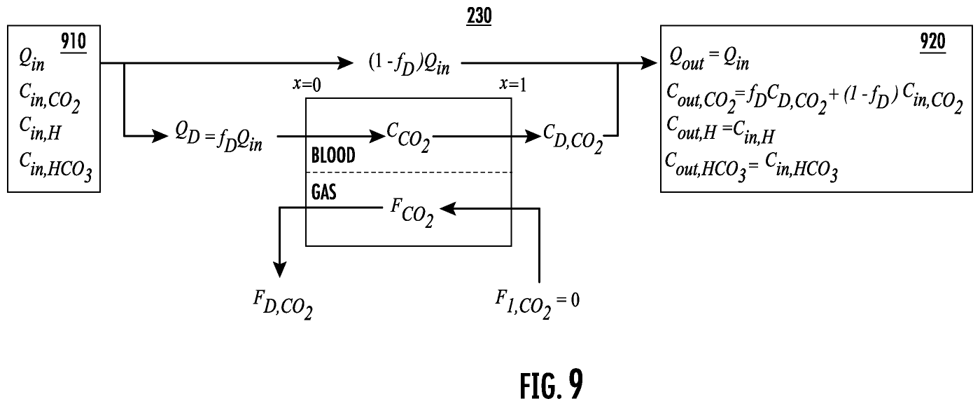

FIG. 9 depicts an illustrative ECCO₂RD model 230 according to some embodiments. In some embodiments, ECCO₂RD model 230 may be approximately modeled as a one-dimensional (1D) diffusion device between blood and air. In various embodiments, ECCO₂RD model 230 may receive input 910 to generate output 920. As shown in FIG. 9, the dynamics of ECCO₂RD model 230 may include the following Equations (22a) and (22b):

$$\frac{\partial {C}_{{CO}_{2}}}{\partial t} = \frac{1}{{V}_{blood}} ( - {Q}_{d} \frac{\partial {C}_{{CO}_{2}}}{\partial x} + κ {𝒟}_{{CO}_{2}} ( {P}_{gas} - {P}_{blood} ) ) \qquad ( 22 a )$$

$$\frac{\partial F}{\partial t} = \frac{1}{{V}_{gas}} ( {\dot{V}}_{D} \frac{\partial F}{\partial x} + {𝒟}_{{CO}_{2}} ( {P}_{blood} - {P}_{gas} ) ) \qquad ( 22 b )$$

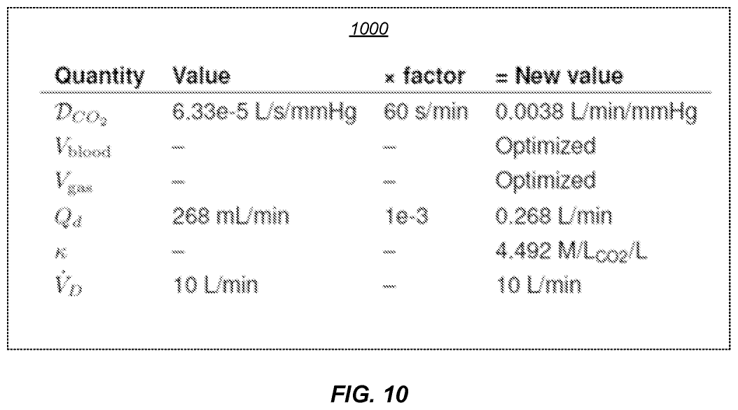

In some embodiments, the equations that describe the dynamics of ECCO₂RD model 230, such as Equations (22a) and (22b), may be the same, similar to, and/or adapted from the equations described in Habran et al., “Mathematical Modeling of Extracorporeal CO2 Removal Therapy,” Medical & Biological Engineering & Computing, 56(3):421-434(2018)(“Habran et al.”), the contents of which are incorporated by reference as if fully set forth in the present disclosure. FIG. 10 depicts Table 1000 providing illustrative values of parameters used in Equations (22a) and (22b):

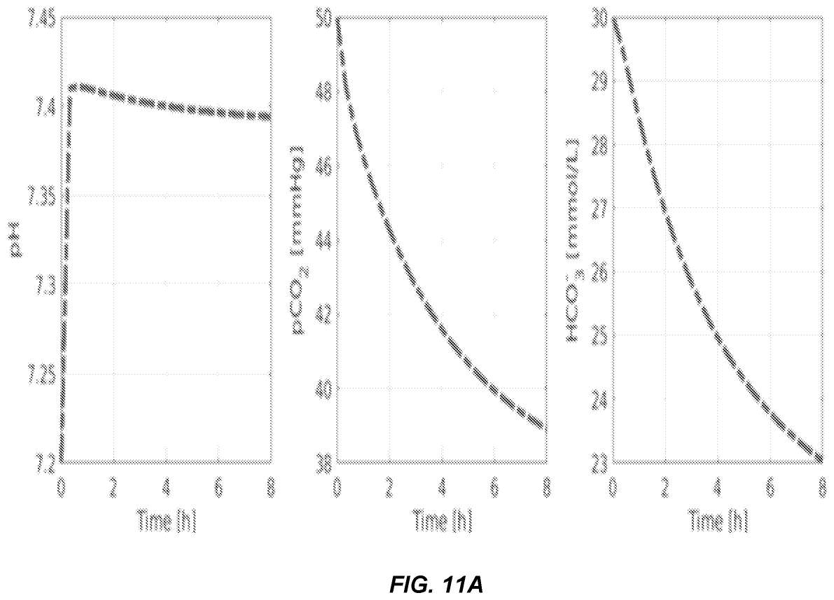

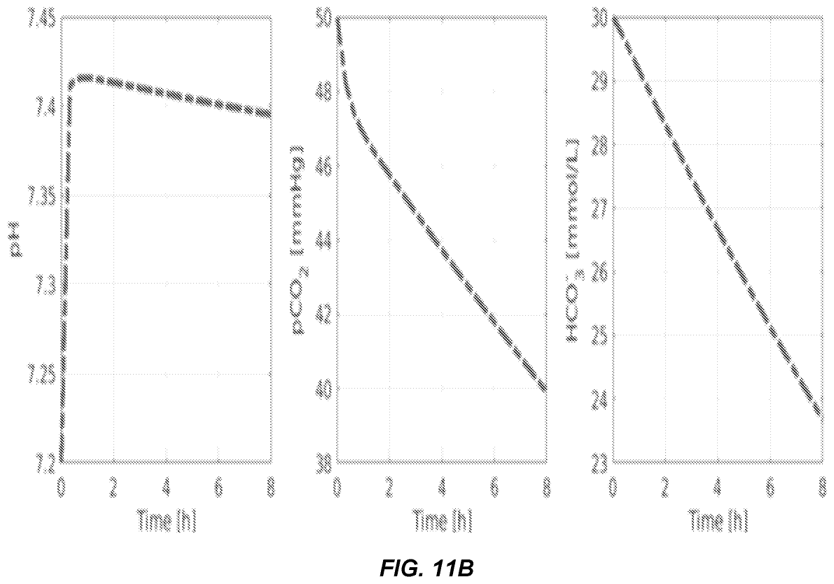

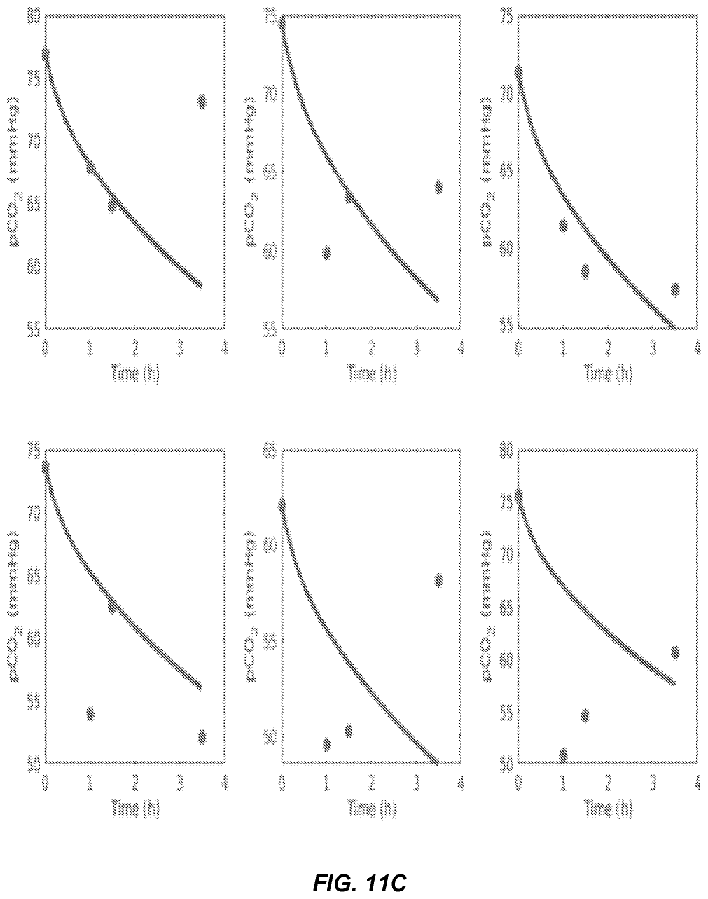

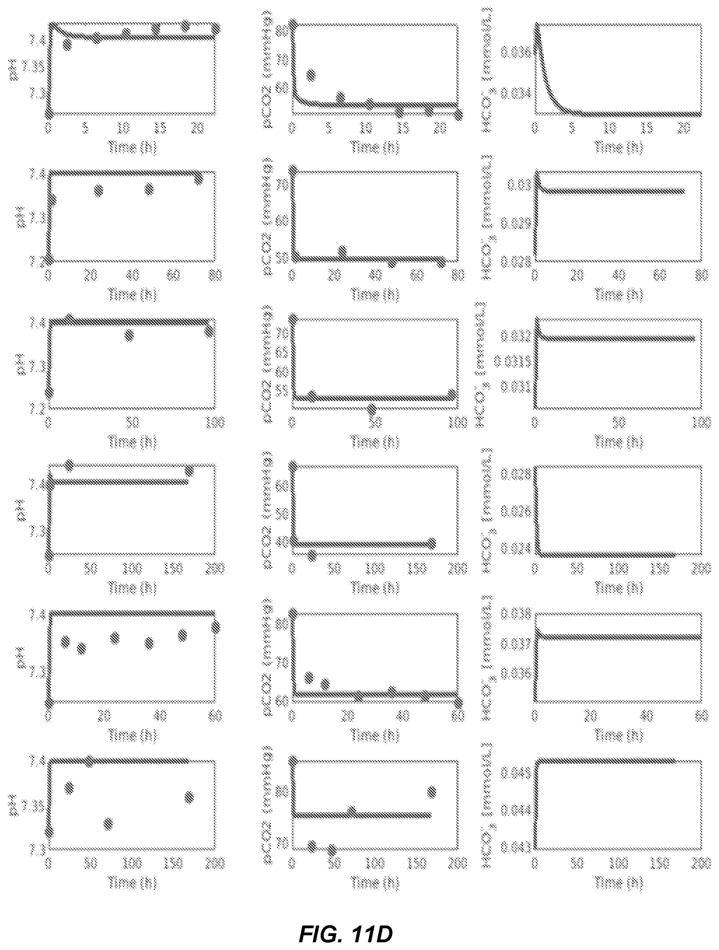

FIGS. 11A-11D depict results for an acid-base model according to some embodiments. More specifically, FIG. 11A depicts results for a patient with normal renal function; FIG. 11B depicts results for a patient without renal function; FIG. 11C depicts results for calculated P_{CO}_{₂} as a function of time over measured points; and FIG. 11D depicts results for time evolution of a model with validation data.

In some embodiments, the results for the ECCO₂RD model may be determined for [symbol] _{d}=268 mL/min, the parameters for the patient may be [symbol] ₚ=350 mL/min, V₀=0 L/min and the patient has full residual kidney function (ρ_{residual}=1). The optimized parameters may be P_{H}=1.6962×10⁻⁷ mol/L/min, P_{CO2}=3.8074×10−6 mol/L/min, D_{CO2}=9.9278×10⁻⁵L−1,γ=2.9864×103 min−1, V_{blood}=0.5294 L, and V_{gas}=2.9694 L.

### EXAMPLE RESULTS: VALIDATION OF ACID-BASE MODELS

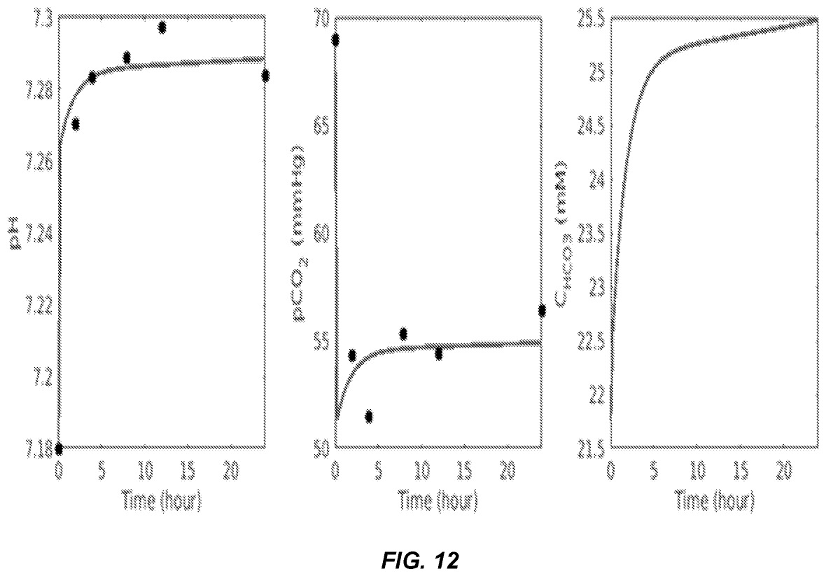

An average of data from 10 ventilated critically ill patients with ARDS and AKI undergoing renal and respiratory replacement therapy according to Forster et al., “Low-flow CO2 removal integrated into a renal-replacement circuit can reduce acidosis and decrease vasopressor requirements,” Crit Care 17, R154 (2013). https://doi.org/10.1186/cc12833 was used to validate the model. The following parameter values were set at the beginning of treatment: blood flow 355±79 ml/min, dialysate flow 2.3±7 L/h, ultrafiltration rate 58±66 ml/h, gas flow 5.2±1.0 L/min, treatment time 95±68 h. The model was parameterized to obtain patient-specific parameters. FIG. 12 depicts results for an acid-base model according to some embodiments, in which the results are depicted for model prediction for a patient with renal failure and low ventilation. In FIG. 12, the solid line is the model prediction; the dots are patient data. As indicated by FIG. 12, models according to some embodiments are capable of accurately predicting the pH, and pCO₂ over time for example, for at least the first 24 hours of treatment.

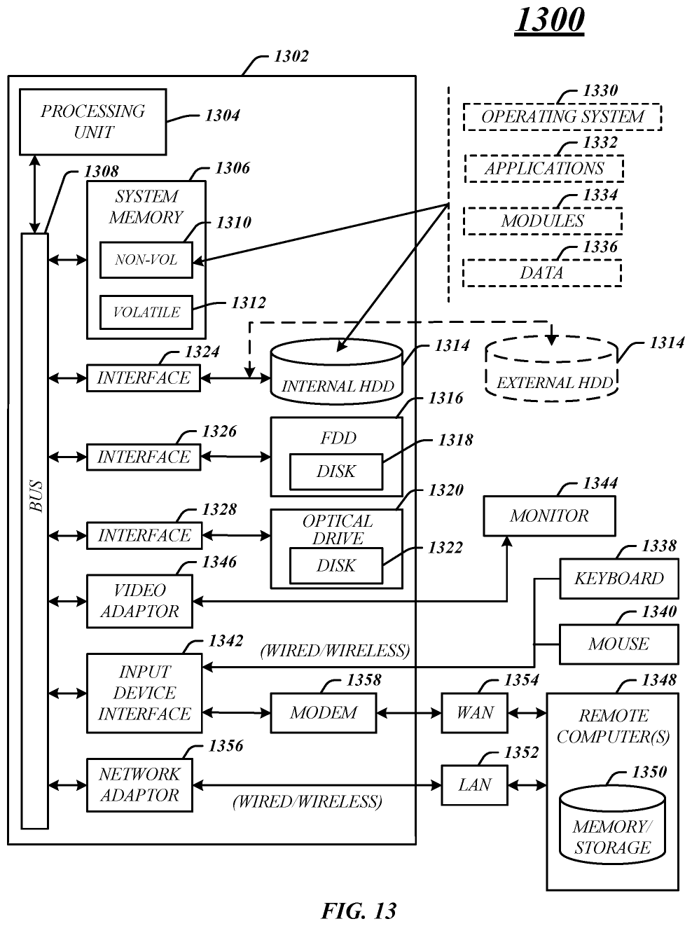

FIG. 13 illustrates an embodiment of an exemplary computing architecture 1300 suitable for implementing various embodiments as previously described. In various embodiments, the computing architecture 1300 may comprise or be implemented as part of an electronic device. The embodiments are not limited in this context. \

As used in this application, the terms “system” and “component” and “module” are intended to refer to a computer-related entity, either hardware, a combination of hardware and software, software, or software in execution, examples of which are provided by the exemplary computing architecture 1300. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 1300 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 1300.

As shown in FIG. 13, the computing architecture 1300 comprises a processing unit 1304, a system memory 1306 and a system bus 1308. The processing unit 1304 may be a commercially available processor and may include dual microprocessors, multi-core processors, and other multi-processor architectures.

The system bus 1308 provides an interface for system components including, but not limited to, the system memory 1306 to the processing unit 1304. The system bus 1308 can be any of several types of bus structure that may further interconnect to a memory bus (with or without a memory controller), a peripheral bus, and a local bus using any of a variety of commercially available bus architectures. Interface adapters may connect to the system bus 1308 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 1306 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (for example, USB memory, solid state drives (SSD) and any other type of storage media suitable for storing information. In the illustrated embodiment shown in FIG. 13, the system memory 1306 can include non-volatile memory 1310 and/or volatile memory 1312. A basic input/output system (BIOS) can be stored in the non-volatile memory 1310. The computer 1302 may include various types of computer-readable storage media in the form of one or more lower speed memory units, including an internal (or external) hard disk drive (HDD) 1314, a magnetic floppy disk drive (FDD) 1316 to read from or write to a removable magnetic disk 1311, and an optical disk drive 1320 to read from or write to a removable optical disk 1322 (for example, a CD-ROM or DVD). The HDD 1314, FDD 1316 and optical disk drive 1320 can be connected to the system bus 1308 by a HDD interface 1324, an FDD interface 1326 and an optical drive interface 1328, respectively. The HDD interface 1324 for external drive implementations can include at least one or both of Universal Serial Bus (USB) and IEEE 1114 interface technologies.

The drives and associated computer-readable media provide volatile and/or nonvolatile storage of data, data structures, computer-executable instructions, and so forth. For example, a number of program modules can be stored in the drives and memory units 1310, 1312, including an operating system 1330, one or more application programs 1332, other program modules 1334, and program data 1336. In one embodiment, the one or more application programs 1332, other program modules 1334, and program data 1336 can include, for example, the various applications and/or components of a computing device.

A user can enter commands and information into the computer 1302 through one or more wired/wireless input devices, for example, a keyboard 1338 and a pointing device, such as a mouse 1340. These and other input devices are often connected to the processing unit 1304 through an input device interface 1342 that is coupled to the system bus 1308, but can be connected by other interfaces.

A monitor 1344 or other type of display device is also connected to the system bus 1308 via an interface, such as a video adaptor 1346. The monitor 1344 may be internal or external to the computer 1302. In addition to the monitor 1344, a computer typically includes other peripheral output devices, such as speakers, printers, and so forth.

The computer 1302 may operate in a networked environment using logical connections via wired and/or wireless communications to one or more remote computers, such as a remote computer 1348. The remote computer 1348 can be a workstation, a server computer, a router, a personal computer, portable computer, microprocessor-based entertainment appliance, a peer device or other common network node, and typically includes many or all of the elements described relative to the computer 1302, although, for purposes of brevity, only a memory/storage device 1350 is illustrated. The logical connections depicted include wired/wireless connectivity to a local area network (LAN) 1352 and/or larger networks, for example, a wide area network (WAN) 1354. Such LAN and WAN networking environments are commonplace in offices and companies, and facilitate enterprise-wide computer networks, such as intranets, all of which may connect to a global communications network, for example, the Internet.

The computer 1302 is operable to communicate with wired and wireless devices or entities using the IEEE 802 family of standards, such as wireless devices operatively disposed in wireless communication (for example, IEEE 802.16 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (for example, electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

1. An apparatus, comprising:
at least one processor; and
a memory coupled to the at least one processor, the memory comprising instructions that, when executed by the at least one processor, cause the at least one processor to:
access an acid-base model configured to model acid-base homeostasis of a patient, the acid-base model comprising a patient model, a dialyzer model, and an extracorporeal CO₂ removal device (ECCO₂RD) model, and
determine predicted patient information using the acid-base model.

2. The apparatus of claim 1, the predicted patient information comprising at least one of a blood flow rate (Q), a serum pH level, a pCO₂ level, or a HCO₃ level.

3. The apparatus of claim 1, the instructions, when executed by the at least one processor, to cause the at least one processor to determine continuous renal replacement therapy (CRRT) parameters to control acid-base status based on the predicted patient information.

4. The apparatus of claim 1, the acid-base model configured to model the regulation of H⁺, CO₂, and HCO₃⁻.

5. The apparatus of claim 1, the patient model configured to model patient physiology having input of blood flow and output of hydrogen ion concentration, carbon dioxide concentration, and bicarbonate concentration.

6. The apparatus of claim 1, the dialyzer model configured to model continuous renal replacement therapy (CRRT).

7. The apparatus of claim 1, the ECCO₂RD model configured to model a one-dimensional (1D) diffusion device between blood and air.

8. The apparatus of claim 1, the acid-base model comprising a blood flow circuit flowing from a patient, modeled by the patient model, to a dialyzer, modeled by the dialyzer mode, to an ECCO2RD, modeled by the ECCO2RD model, and back to the patient.

9. The apparatus of claim 1, the blood circuit comprising diffusion at any point in the blood circuit.

10. The apparatus of claim 1, the patient comprising a virtual patient.

11. The apparatus of claim 1, the predicted patient information comprising a treatment recommendation.

12. The apparatus of claim 11, the treatment recommendation comprising a treatment process for an acid-base disorder.

13. A computer-implemented method of acid-base homeostasis analysis, the method comprising, via a processor of a computing device:
providing an acid-base model configured to model acid-base homeostasis of a patient, the acid-base model comprising a patient model, a dialyzer model, and an extracorporeal CO₂ removal device (ECCO₂RD) model, and
determining predicted patient information using the acid-base model.

14. The computer-implemented method of claim 13, the predicted patient information comprising at least one of a blood flow rate (Q) , a serum pH level, a pCO₂ level, or a HCO₃ level.

15. The computer-implemented method of claim 13, further comprising prescribing continuous renal replacement therapy (CRRT) parameters to control acid-base status based on the predicted patient information.

16. The computer-implemented method of claim 13, the acid-base model configured to model the regulation of H⁺, CO₂ and HCO₃_{^{−}}.

17. The computer-implemented method of claim 13, the patient model configured to model patient physiology having input of blood flow and output of hydrogen ion concentration, carbon dioxide concentration and bicarbonate concentration.

18. The computer-implemented method of claim 13, the dialyzer model configured to model continuous renal replacement therapy (CRRT).

19. The computer-implemented method of claim 13, the ECCO2RD model configured to model a one-dimensional (1D) diffusion device between blood and air.

20. The computer-implemented method of claim 13, the acid-base model comprising a blood flow circuit flowing from a patient, modeled by the patient model, to a dialyzer, modeled by the dialyzer mode, to an ECCO2RD, modeled by the ECCO2RD model, and back to the patient.
